(19) United States
(12) Patent Application Publication
(10) Pub. No.: US 2007/0078376 A1
(43) Pub. Date: Apr. 5, 2007

(54) Functionalized microneedles transdermal drug delivery systems, devices, and methods

(76) Inventor: Gregory A. Smith, Issaquah, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/520,855

(22) Filed: Sep. 12, 2006

Related U.S. Application Data
(60) Provisional application No. 60/722,789, filed on Sep. 30, 2005.

(51) Int. Cl. A61N 1/30

(52) U.S. Cl. 604/21; 604/171; 604/46

(57) ABSTRACT

Systems, devices, and methods for transdermal delivery of one or more therapeutic active agents to a biological interface. A transdermal drug delivery system is operable for delivering of one or more therapeutic active agents to a biological interface. The system includes an active electrode assembly, a counter electrode assembly, and a plurality of functionalized microneedles.

### CROSS-REFERENCE TO RELATED APPLICATION

This application claims the benefit under 35 U.S.C. §119(e) of U.S. Provisional Patent Application No. 60/722,789 filed Sep. 30, 2005, the contents of which are incorporated herein by reference in their entirety.

### BACKGROUND

1. Field

This disclosure generally relates to the field of iontophoresis and, more particularly, to functionalized microneedles transdermal drug delivery systems, devices, and methods for delivering one or more active agents to a biological interface.

2. Description of the Related Art

Iontophoresis employs an electromotive force and/or current to transfer an active agent (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their ability to deliver drugs through, for example, tissue barriers. For example, it may be desirable to have novel approaches for overcoming the poor permeability of skin.

The present disclosure is directed to overcome one or more of the shortcomings set forth above, and provide further related advantages.

### BRIEF SUMMARY

In one aspect, the present disclosure is directed to a transdermal drug delivery system for delivering of one or more therapeutic active agents to a biological interface. The system includes a surface functionalized substrate having a first side and a second side opposing the first side. The surface functionalized substrate includes a plurality of microneedles projecting outwardly from the first side. Each microneedle includes an outer surface and an inner surface that forms a channel. The channel is operable for providing fluidic communication between the first and the second sides of the surface functionalized substrate. At least one of the inner surface or the outer surface of the microneedles includes one or more functional groups.

In another aspect, the present disclosure is directed to a microneedle structure. The microneedle structure includes a substrate having an exterior and an interior surface, a first side, and a second side opposing the first side. The microneedle structure further includes a plurality of microneedles projecting outwardly from the first side of the substrate. Each microneedle includes a proximate end, a distal end, an outer surface, and an inner surface forming a channel exiting between the proximate and the distal ends to provided fluid communication there between. In some embodiments, at least the inner surface of the microneedles is modified with one or more functional groups.

In yet another aspect, the present disclosure is directed to a method of forming an iontophoretic drug delivery device for providing transdermal delivery of one or more therapeutically active agents to a biological interface. The method includes forming a plurality of hollow microneedles, having an interior and an exterior surface, on a substrate having a first side and a second side opposing the first side, the plurality of hollow microneedles substantially formed on the first side of the substrate. The method further includes functionalizing at least the interior surface of the plurality of hollow microneedles to include one or more functional groups. In some embodiments, the method further includes physically coupling the substrate to an active electrode assembly, the active electrode assembly including at least one active agent reservoir and at least one active electrode element, the at least one active agent reservoir in fluidic communication with the plurality of hollow microneedles, the at least one active electrode element operable to provide an electromotive force to drive an active agent from the at least one active agent reservoir, through the plurality of hollow microneedles, and to the biological interface.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top, front view of a transdermal drug delivery system according to one illustrated embodiment.

FIG. 1B is a top, plan view of a transdermal drug delivery system according to one illustrated embodiment.

FIG. 2A is a bottom, front view of a plurality of microneedles in the form of an array according to one illustrated embodiment.

FIG. 2B is a bottom, front view of a plurality of microneedles in the form of one or more arrays according to another illustrated embodiment.

FIG. 3A is a bottom, front view of a portion of a microneedle structure according to one illustrated embodiment.

FIG. 3B is a bottom, plan view of a portion of a microneedle structure according to one illustrated embodiment.

FIGS. 4A through 4F are vertical, cross-sectional views of a plurality of microneedles according to another illustrated embodiment.

FIGS. 5A and 5B are vertical, cross-sectional views of a microneedle including one or more functionalized surfaces according to some illustrated embodiments.

FIG. 5C is a vertical, cross-sectional view of a microneedle including one or more functional groups in the form of bonded cations according to some illustrated embodiments.

FIG. 5D an exploded view of the microneedle in FIG. 5C including one or more functional groups in the form of bonded amino groups according to another illustrated embodiment.

FIG. 6A is a vertical, cross-sectional view of a microneedle including one or more functionalized surfaces according to some illustrated embodiments.

FIG. 6B an exploded view of the microneedle in FIG. 6A including one or more functionalized groups in the form of polysilanes according to another illustrated embodiment.

FIG. 7 is a synthesis schematic for a sol-gel deposition of alkoxysilane on a substrate according to one illustrated embodiment.

FIGS. 8A is a vertical, cross-sectional view of a microneedle including one or more functional groups in the form of bonded hydroxyl groups according to another illustrated embodiment.

FIGS. 8B is an exploded view of a microneedle including one or more functional groups in the form of bonded hydroxyl groups and lipid groups according to another illustrated embodiment.

FIG. 9 is a schematic diagram of the iontophoresis device of FIGS. 1A and 1B comprising an active and counter electrode assemblies and a plurality of microneedles according to one illustrated embodiment.

FIG. 10 is a schematic diagram of the iontophoresis device of FIG. 9 positioned on a biological interface, with an optional outer release liner removed to expose the active agent, according to another illustrated embodiment.

FIG. 11 is a flow diagram of a method of forming an iontophoretic drug delivery device for providing transdermal delivery of one or more therapeutic active agents to a biological interface according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearance of the phrases “in one embodiment,” or “in an embodiment,” or “another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an electrode element” includes a single electrode element, or two or more electrode elements. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electroosmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

In some embodiments, one or more active agents may be selected form analgesics, anesthetics, anesthetics vaccines, antibiotics, adjuvants, immunological adjuvants, immunogens, tolerogens, allergens, toll-like receptor agonists, toll-like receptor antagonists, immuno-modulators, immuno-response agents, immuno-stimulators, specific immuno-stimulators, non-specific immuno-stimulators, and immuno-suppressants, or combinations thereof.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and a mixture thereof.

As used herein and in the claims, the term “subject” generally refers to any host, animal, vertebrate, or invertebrate, and includes fish, mammals, amphibians, reptiles, birds, and particularly humans.

As used herein and in the claims, the term “functional group” generally refers to a chemical group that confers special properties or particular functions to an article (e.g., a surface, a molecule, a substance, a particle, nanoparticle, and the like). Among the chemical groups, examples include an atom, an arrangement of atoms, an associated group of atoms, molecules, moieties, and that like, that confer certain characteristic properties on the article comprising the functional groups. Exemplary characteristic properties and/or functions include chemical properties, chemically reactive properties, association properties, electrostatic interaction properties, bonding properties, biocompatible properties, and the like. In some embodiments, the functional groups include one or more nonpolar, hydrophilic, hydrophobic, organophilic, lipophilic, lipophobic, acidic, basic, neutral, functional groups, and the like.

As used herein and in the claims, the term “functionalized surface” generally refers to a surface that has been modified so that a plurality of functional groups is present thereon. The manner of treatment is dependent on, for example, the nature of the chemical compound to be synthesized and the nature and composition of the surface. In some embodiments, the surface may include functional groups selected to impart one or more of properties to the surface including nonpolar, hydrophilic, hydrophobic, organophilic, lipophilic, lipophobic, acidic, basic, neutral, properties, increased or decreased permeability, and the like, and/or combinations thereof.

As used herein and in the claims, the term “frustum” or “frusta” generally refers to any structure having an axial cross-section that generally decreases. Frusta structures can have a cross-section that decreases discontinuously or generally continuously from an upper end to a lower end. Typical frusta generally include a wide end and a narrow end. For example, a pyramidal frustum may resemble a pyramid missing its apical portion. In some embodiments, the term “frustum” includes structures having a cross-section of substantially any shape including circular, triangular, square, rectangular polygonal, and the like, as well as other symmetrical and asymmetrical shapes. A frustum may further include substantially conical structures, and frusto-conical structures, as well as faceted structures including prismatoids, polyhedrons, pyramids, prisms, wedges, and the like.

The headings provided herein are for convenience only and do not interpret the scope or meaning of the embodiments.

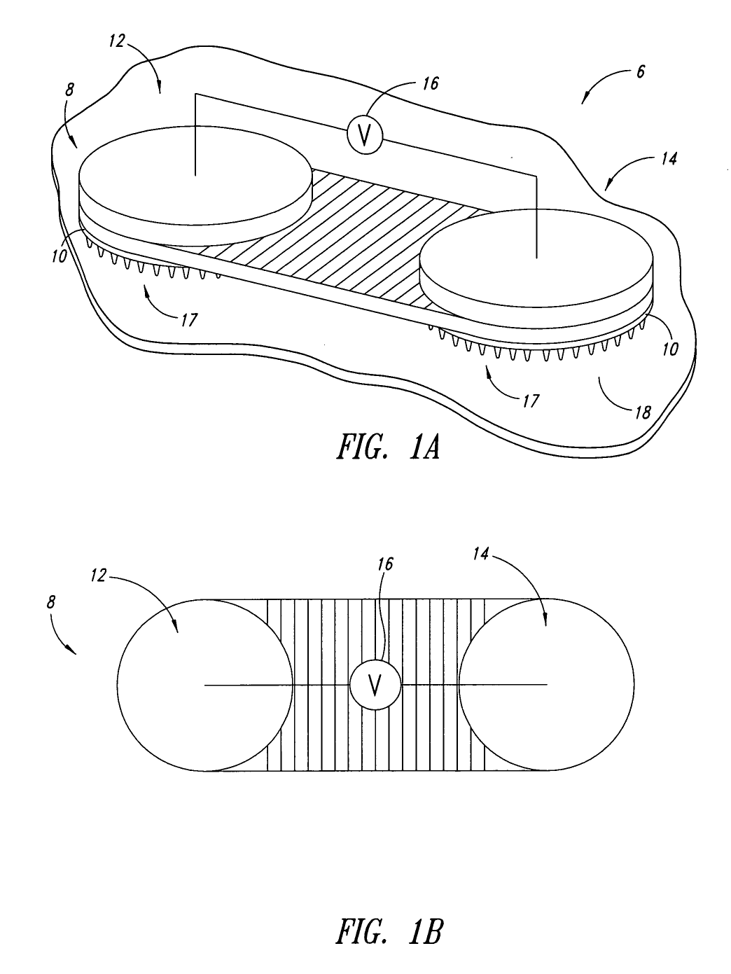

FIGS. 1A and 1B show an exemplary transdermal drug delivery system 6 for delivering of one or more active agents to a subject. The system 6 includes an iontophoresis device 8 including active and counter electrode assemblies 12, 14, respectively, and an integrated power source 16, and one or more surface functionalized substrates 10 including a plurality of microneedles 17. The active and counter electrode assemblies 12, 14, are electrically coupleable to the integrated power source 16 to supply an active agent contained in the active electrode assembly 12, via iontophoresis, to a biological interface 18 (e.g., a portion of skin or mucous membrane).

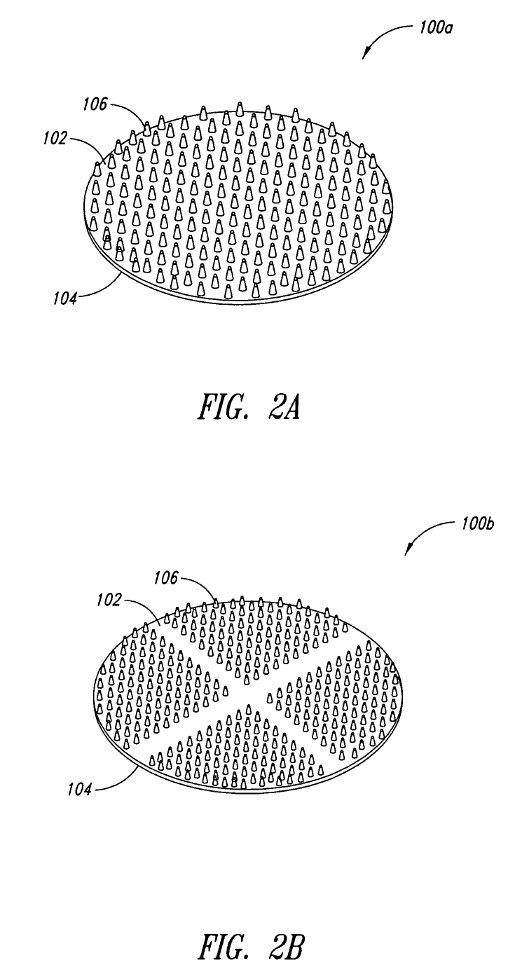

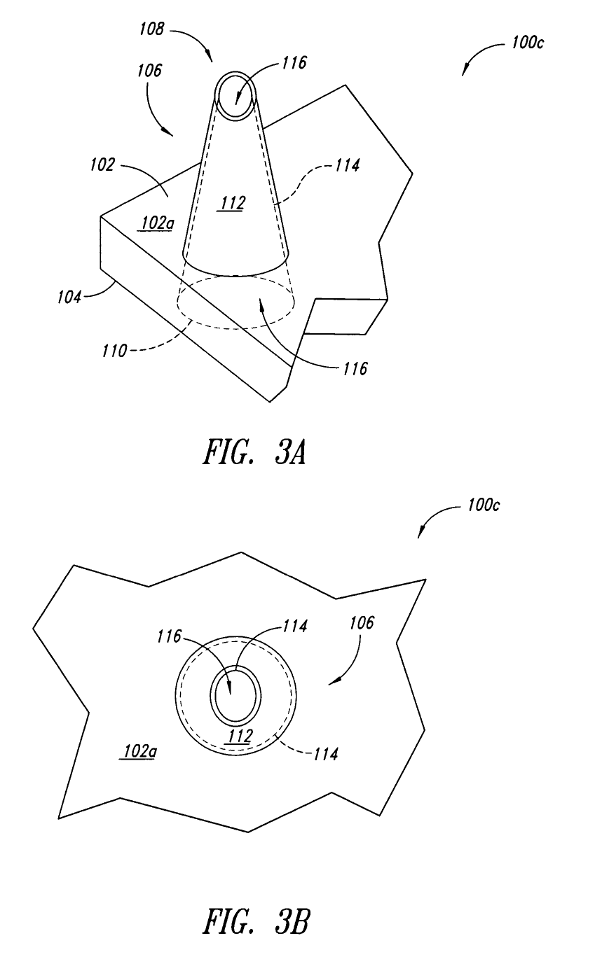

As shown in FIGS. 2A, 2B, 3A, and 3B, the surface functionalized substrate 10 includes a first side 102 and a second side 104 opposing the first side 102. The first side 102 of the surface functionalized substrate 10 includes a plurality of microneedles 106 projecting outwardly from the first side 102 of the surface functionalized substrate 10. The surface functionalized substrate 10 can comprise any material suitable for fabricating microneedles 106 including ceramics, elastomers, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon-based organic polymers, silicon rubbers, superconducting materials (e.g., superconducting wafers, and the like), and the like, as well as combinations, composites, and/or alloys thereof. The surface functionalized substrate 10 may take any geometric form including, circular, triangular, square, rectangular, polyhedral, regular or irregular forms, and the like. In an embodiment, the surface functionalized substrate 10 include at least one material selected from ceramics, metals, polymers, molded plastics, superconducting wafers, and the like, as well as combinations, composites, and/or alloys thereof.

With particular reference to FIGS. 3A and 3B, in some embodiments, substrate 10 takes the form of a microneedle structure 100c. The microneedle structure 100c includes a substrate 10 having an exterior 102a and an interior surface 104a, a first side 102, and a second side 104 opposing the first side 102. The microneedle structure 100c further includes a plurality of microneedles 106 (one shown in FIGS. 3A and 3B) projecting outwardly from the first side 102 of the substrate 10. Each microneedle 106 includes a proximate 110 and a distal end 108, an outer surface 112 and an inner surface 114 forming a channel 116 exiting between the proximate and the distal ends 110,108, respectively, to provided fluid communication there between. In some embodiments, at least the inner surface 114 of the microneedles is modified with one or more functional groups. In some other embodiments, at least the interior surface 104a of the substrate is modified with a sufficient amount of one or more functional groups. In some embodiments, each microneedle 106 is substantially hollow, and each microneedle 106 is substantially in the form of a frusto-conical annulus. In some further embodiments, the plurality of microneedles 106 is integrally formed from the substrate 10.

The microneedles 106 may be individually provided or formed as part of one or more arrays 100a, 100b (FIGS. 2A and 2B). In some embodiments, the microneedle 106 are integrally formed from the substrate 10. The microneedles 106 may take a solid and permeable form, a solid and semi-permeable form, and/or a solid and non-permeable form. In some other embodiments, solid, non-permeable, microneedles may further comprise grooves along their outer surfaces for aiding the transdermal delivery of one or more active agents. In some other embodiments, the microneedles 106 may take the form of hollow microneedles (as show in, for example, FIGS. 3A and 3B). In some embodiments, the hollow microneedles may be filled with ion exchange material, ion selective materials, permeable materials, semi-permeable materials, solid materials, and the like.

The microneedles 106 are used, for example, to deliver a variety of pharmaceutical compositions, molecules, compounds, active agents, and the like to a living body via a biological interface, such as skin or mucous membrane. In certain embodiments, pharmaceutical compositions, molecules, compounds, active agents, and the like may be delivered into or through the biological interface. For example, in delivering pharmaceutical compositions, molecules, compounds, active agents, and the like via the skin, the length of the microneedle 106, either individually or in arrays 100a, 100b, and/or the depth of insertion may be used to control whether administration of a pharmaceutical compositions, molecules, compounds, active agents, and the like is only into the epidermis, through the epidermis to the dermis, or subcutaneous. In certain embodiments, the microneedle 106 may be useful for delivering high-molecular weight active agents, such as those comprising proteins, peptides and/or nucleic acids, and corresponding compositions thereof. In certain embodiments, for example wherein the fluid is an ionic solution, the microneedles 106 can provide electrical continuity between the power source 16 and the tip of the microneedle 106. In some embodiments, the microneedles 106, either individually or in arrays 100a, 100b, may be used to dispense, deliver, and/or sample fluids through hollow apertures, through the solid permeable or semi permeable materials, or via external grooves. The microneedles 106 may further be used to dispense, deliver, and/or sample pharmaceutical compositions, molecules, compounds, active agents, and the like by iontophoretic methods, as disclosed herein.

Accordingly, in certain embodiments, for example, a plurality of microneedles 106 in an array 100a, 100b may advantageously be formed on an outermost biological interface-contacting surface of an transdermal drug delivery system 6. In some embodiments, the pharmaceutical compositions, molecules, compounds, active agents, and the like delivered or sampled by such a system 6 may comprise, for example, high-molecular weight active agents, such as proteins, peptides, and/or nucleic acids.

As shown in FIGS. 2A and 2B, in some embodiments, a plurality of microneedles 106 may take the form of a microneedle array 100a, 100b. The microneedle array 100a, 100b may be arranged in a variety of configurations and patterns including, for example, a rectangle, a square, a circle (as shown in FIG. 2A), a triangle, a polygon, a regular or irregular shapes, and the like. The microneedles 106 and the microneedle arrays 100a, 100b may be manufactured from a variety of materials, including ceramics, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon rubbers, silicon-based organic polymers, superconducting materials (e.g., superconducting wafers, and the like), and the like, as well as combinations, composites, and/or alloys thereof. Techniques for fabricating the microneedles 106 are well known in the art and include, for example, electro-deposition, electro-deposition onto laser-drilled polymer molds, laser cutting and electro-polishing, laser micromachining, surface micro-machining, soft lithography, x-ray lithography, LIGA techniques (e.g., X-ray lithography, electroplating, and molding), injection molding, conventional silicon-based fabrication methods (e.g., inductively coupled plasma etching, wet etching, isotropic and anisotropic etching, isotropic silicon etching, anisotropic silicon etching, anisotropic GaAs etching, deep reactive ion etching, silicon isotropic etching, silicon bulk micromachining, and the like), complementary-symmetry/metal-oxide semiconductor (CMOS) technology, deep x-ray exposure techniques, and the like. See for example, U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311. Some or all of the teachings therein may be applied to microneedle devices, their manufacture, and their use in iontophoretic applications. In some techniques, the physical characteristics of the microneedles 106 depend on, for example, the anodization conditions (e.g., current density, etching time, HF concentration, temperature, bias settings, and the like) as well as substrate properties (e.g., doping density, doping orientation, and the like).

As show in FIGS. 3A and 3B, in some embodiments, each microneedle 106 includes a proximate end 108, a distal end 110, an outer surface 112, and an inner surface 114. The inner surface 114 of microneedle 106 forms a channel 116 that exits between the proximate and distal ends 108, 110 to provided fluid communication there between. The outer surface 112 of the plurality of microneedles 106 comprises a portion of the first side 102 of surface functionalized substrate 10, and the inner surface 114 of the plurality of microneedles 106 comprises a portion of the second side 104 of surface functionalized substrate 10.

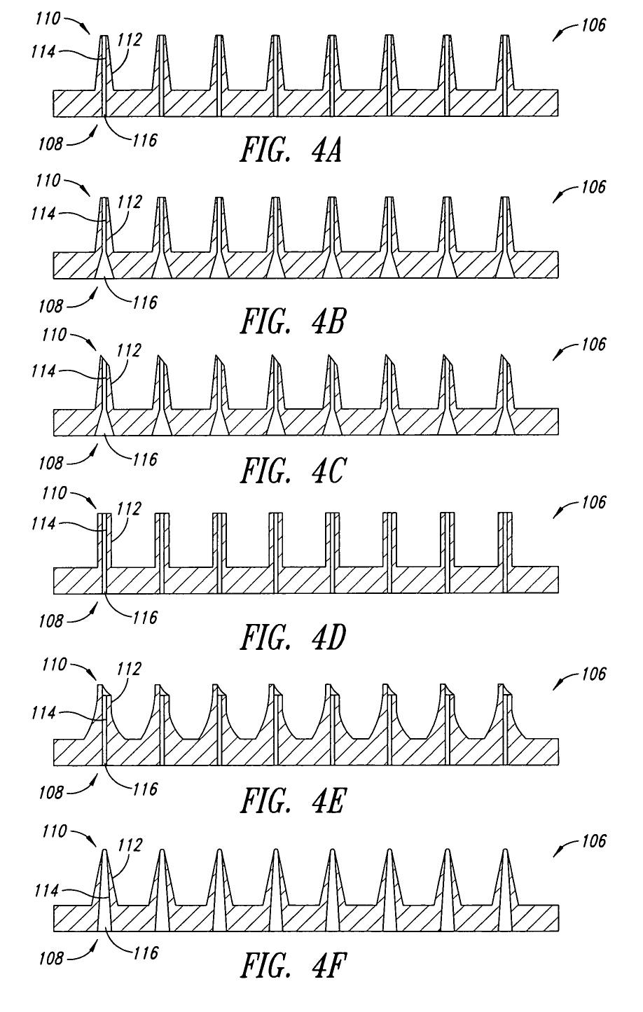

As shown in FIGS. 4A through 4F, the distal end 110, the outer surface 112, and the inner surface 114 may each take a variety of shapes and forms. For example, the distal end 110 of the microneedle 106 may be sharp or dull, and may take a beveled, parabolic, flat-tipped, sharp-tip, blunt-tipped, radius-tipped, chisel-like, tapered, and/or tapered-cone-like form. The outer shape of the microneedles including the outer surface 112 may take any form including a right cylinder, an oblique cylinder, a circular cylinder, a polygonal cylinder, a frustum, an oblique frustum, a regular or irregular shape, and the like.

The channel 116 formed by the inner surface 114 may take any form including a right cylinder, an oblique cylinder, a circular cylinder, a polygonal cylinder, a frustum, an oblique frustum and the like. The channel 116 may also take the form of a regular or irregular shape as long as it is operable to provide fluid communication between the distal and proximate ends 110, 112 of the microneedle 106. In some embodiments, the plurality of microneedles 106 may take the form of hollow microcapillaries.

The microneedles 106 may be sized and shaped to penetrate the outer layers of skin to increase its permeability and transdermal transport of pharmaceutical compositions, molecules, compounds, active agents, and the like. In some embodiments, the microneedles 106 are sized and shaped with an appropriate geometry and sufficient strength to insert into a biological interface (e.g., the skin or mucous membrane on a subject, and the like), and thereby increase a trans-interface (e.g., transdermal) transport of pharmaceutical compositions, molecules, compounds, active agents, and the like.

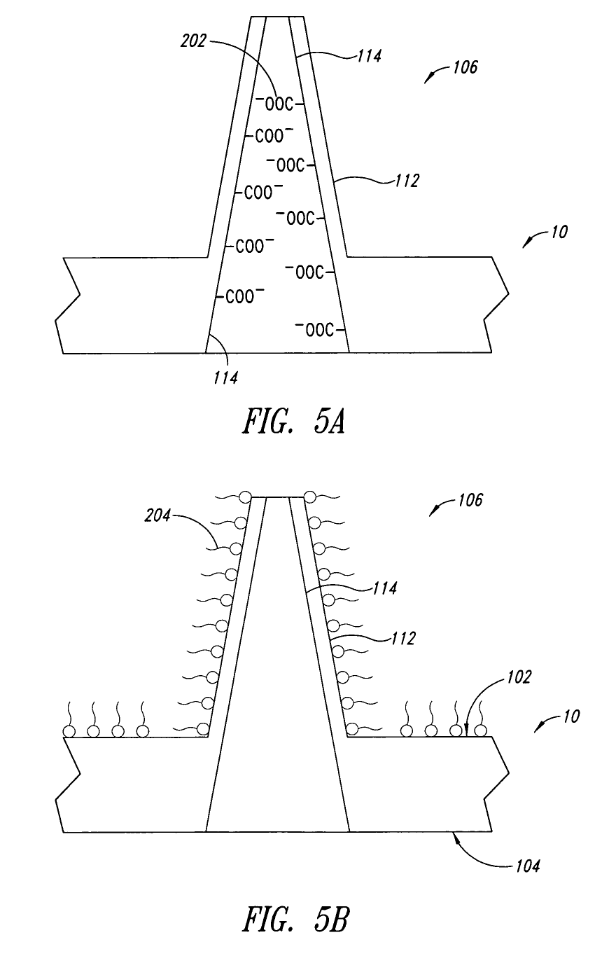

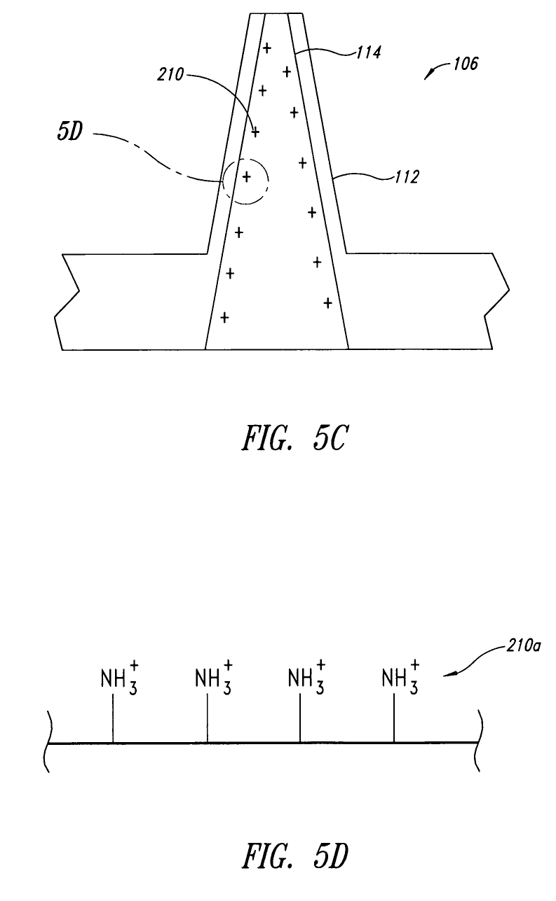

As previously noted, the outer surface 112 of the plurality of microneedles 106 comprises a portion of the first side 102 of the surface functionalized substrate 10, and the inner surface 114 of the plurality of microneedles 106 comprises a portion of the second side 104 of the surface functionalized substrate 10. As shown in FIGS. 5A-5D, 6A, 6B, 8A, and 8B, either the outer surface 112, or the inner surface 114, or both may be modified to include one or more functional groups. In some embodiments, at least a portion of either the outer surface 112, or the inner surface 114, or both may be modified to include one or more functional groups. In some other embodiments, at least the interior surface 114 of the substrate 10 is modified with a sufficient amount of one or more functional groups. Examples of functional groups include, charge functional groups, hydrophobic functional groups, hydrophilic functional groups, chemically reactive functional groups, organofunctional group, water-wettable groups, bio-compatible groups, and the like. In some embodiments, the functional groups may be selected to impart one or more properties to the surface functionalized substrate 10 selected from, for example, nonpolar, hydrophilic, hydrophobic, organophilic, lipophilic, lipophobic, acidic, basic, neutral, properties, increased or decreased permeability, and the like, and/or combinations thereof. Certain functional groups may impart one or more properties to the surface functionalized substrate 10, and may comprise one or more functionalities (e.g., charge functionally, hydrophobic functionally, hydrophilic functionally, chemically reactive functionally, organo functionally, water-wettable functionally, and the like).

Among the functional groups examples include alcohols, hydroxyls, amines, aldehydes, dyes, ketones, cabonyls, thiols, phosphates, carboxyls, caboxilyic acids, carboxylates, proteins, lipids, polysaccharides, pharmaceuticals, metals, —NH₃⁺, —COOH, —COO—, —SO₃, —CH₂N⁺(CH₃)₃, —(CH₂)ₘCH₃, —C((CH₂)ₘCF₃)₃, —CH₂N(C₂H₅)₂, —NH₂, —(CH₂)ₘCOOH, —(OCH₂CH₂)ₘCH₃, —SiOH, —OH, and the like.

In some embodiments, the functional groups are selected form Formula I alkoxysilanes:
(R²)Si(R¹)₃ (Formula I)
wherein R¹ is selected from a chlorine, an acetoxy, and an alkoxy, and R² is selected from an organofunctional group, an alkyl, an aryl, an amino, a methacryloxy, and an epoxy.

In some embodiments, the functional groups may include a binding group (e.g., coupling agents, and the like), a linking group (e.g., spacer groups, organic spacer groups, and the like), and/or a matrix-forming group that aid in, for example, binding the functional groups to the surface functionalize substrate 10, or aid in providing the desired functionality. Examples of binding groups are well known in the art and include acrylates, alkoxysilanes, alkyl thiols, arenes, azidos, carboxylates, chlorosilanes, alkoxysilanes, acetocysilanes, silazanes, disilazanes, disulfides, epoxides, esters, hydrosilyl, isocyanates. and phosphoamidites, isonitriles, methacrylates, nitrenes, nitriles, quinones, silanes, sulfhydryls, thiols, vinyl groups, and the like. Examples of linking groups are well known in the art and include dendrimers, polymers, hydrophilic polymers, hyperbranched polymers, poly(amino acids), polyacrylamides, polyacrylates, polyethylene glycols, polyethylenimines, polymethacrylates, polyphosphazenes, polysaccharides, polysiloxanes, polystyrenes, polyurethanes, propylene's, proteins, telechelic block copolymers, and the like. Examples of matrix-forming groups are well known in the art and include dendrimer polyamine polymers, bovine serum albumin, casein, glycolipids, lipids, heparins, glycosaminoglycans, muscin, surfactants, polyoxyethylene-based surface-active substances (e.g., polyoxyethlene-polyoxypropylene copolymers, polyoxyethylene 12 tridecyl ether, polyoxyethylene 18 tridecyl ether, polyoxyethylene 6 tridecyl ether, polyoxyethylene sorbitan tetraoleate, polyoxyethylene sorbitol hexaoleate, and the like) polyethylene glycols, polysaccharides, serum dilutions, and the like.

As shown in FIG. 5A, the inner surface 114 of the microneedle 106 may be modified to include one or more functional groups. For example, the inner surface 114 may include one or more carboxylic groups 202 capable of imparting the inner surface 114 of the microneedle 106 with a more hydrophilic, anionic surface.

As shown in FIG. 5B, the outer surface 112 of the microneedle 106 may be modified to include one or more functional groups. For example, the outer surface 112 may include one or more lipid groups 204 capable of imparting the outer surface 112 of the microneedle 106 with a more hydrophobic, lipophilic surface. In some embodiments, the lipid groups 204 are deposited directly on the substrate 10 (e.g., as solid-supported membranes). In some other embodiments, the substrate 10 is modified with lipid groups 204 using an ultra-thin polymer supports (e.g., polymer-supported membranes). In some other embodiments, the substrate 10 is modified with lipid groups 204 using well known thiol deposition techniques.

As shown in FIGS. 5C and 5D, the inner surface 114 may include one or more amino groups 202 capable of imparting the inner surface 114 of the microneedle 106 with a more hydrophilic, cationic surface.

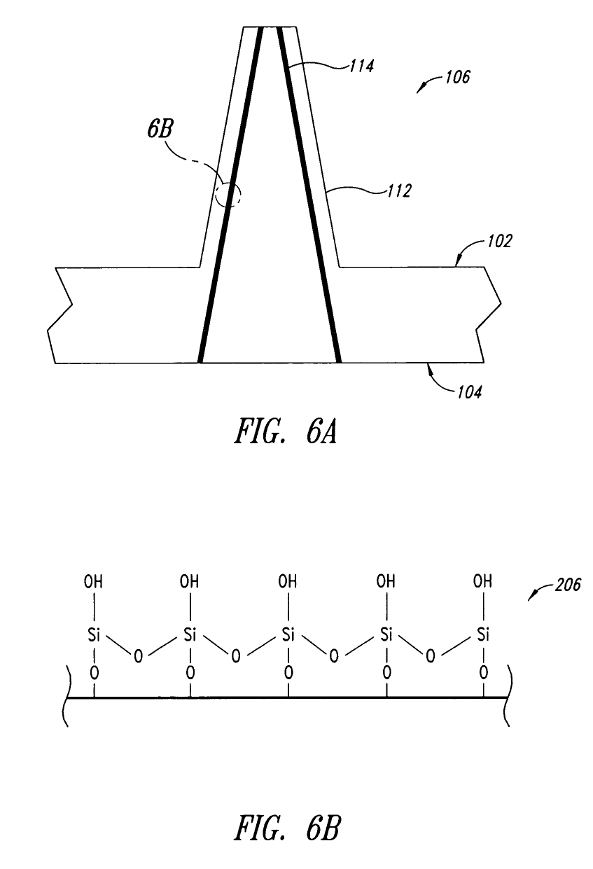

As shown in FIGS. 6A and 6B, in some embodiments, at least a portion of either the outer surface 112, or the inner surface 114, or both may be modified to include one or more silane groups. For example, the inner surface 114 may be modified to include one or more Formula I alkoxysilanes:
(R²)Si(R¹)₃ (Formula I)
wherein R¹ is selected from a chlorine, an acetoxy, and an alkoxy, and R² is selected from an organofunctional group (e.g., methyl, phenyl, isobutyl, octyl, —NH(CH₂)₃NH₂, epoxy, methacryl, and the like), an alkyl, an aryl, an amino, a methacryloxy, and an epoxy.

Depending on the R¹ and/or R² substituents, the Formula I silanes may impart one or more properties to the surface functionalized substrate 10 selected from, for example, nonpolar, hydrophilic, hydrophobic, organophilic, lipophilic, lipophobic, acidic, basic, neutral, properties, increased or decreased permeability, and the like, and/or combinations thereof. Protocols for functionalizing the surfaces of substrates 10 are well known in the art and include, for example, sol-gel deposition of silanes, silanation, chemical grafting of surface polymers, surface plating, oxidation, plasma deposition, e-beam, sputtering, and the like.

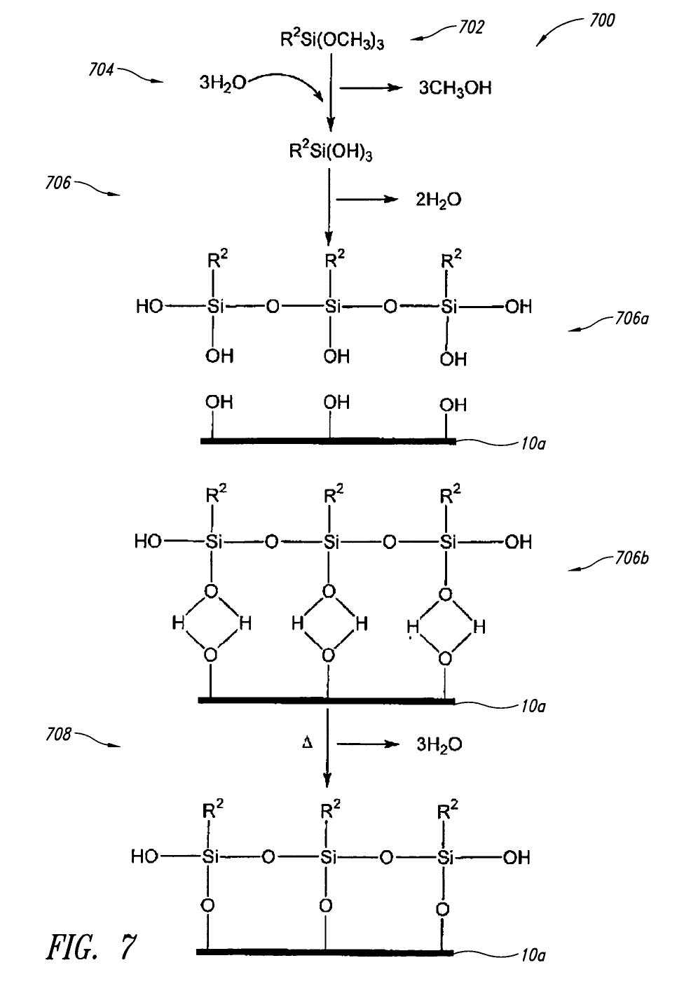

As shown in FIG. 7, one such protocol 700 includes Formula I silanes 702 to modify the physical and chemical prosperities of a substrate 10a comprising one or more hydroxyl functional groups 704. Through controlled hydrolysis 704 and polycondensation 706 of the silanes 702, it is possible to functionalize the surface of the substrate 10a with a polymeric network of, for example, alkoxysilanes 708.

The protocol 700 commences with the hydrolysis 704 of Formula I silanes 702 with water to form alcohol and silanols 704a. The silanols 704a undergo condensation to form polysilanols 706a. The polysilanols 706a can subsequently form hydrogen bonds with the surface of the substrate 10a. Heating 708 causes the hydrogen-bonded polysilanols 706b to lose water and further form covalent bonds with the resulting surface functionalized substrate 10a.

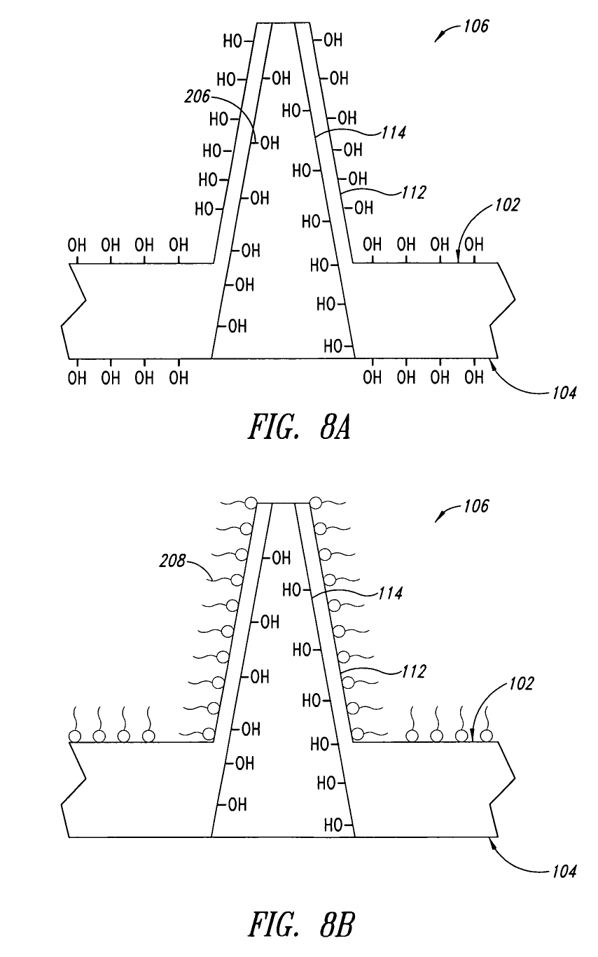

As shown in FIGS. 8A and 8B, in some embodiments, either the first side 102 of the surface functionalized substrate 10 including the outer surface 112 of the microneedles 106, or the second side 104 of the surface functionalized substrate 10 including the inner surface 114 of the microneedles 106, or both may be modified to include one or more functional groups. For example, both the first and second sides 102, 104 of the surface functionalized substrate 10 may be modified with the same functional group (as shown in FIG. 8A). In some embodiments, the first side 102 may comprise a different functional group than the second side 104 (as shown in FIG. 8B).

In some embodiments, the functional groups are selected from charge functional groups capable of maintaining either a positive or negative charge over a broad range of environments (e.g., varying pH range). Examples of charge functional groups include cations, anions, amines, acids, halocarbons, sulfonic acids, quaternary amines, metals, —NH₃⁺, —COOH, —COO⁻, —SO₃, —CH₂N⁺(CH₃)₃, and the like.

In some embodiments, the functional groups are selected from water-wettable groups capable of imparting a surface with the ability to retain a substantially unbroken film of water thereon. For example, at least a portion of the substrate 10 may be modified with water-wettable groups selected from —SiOH, —OH, and the like.

As shown in Figures in 5B, 8A, and 8B, in some embodiments, either the first side 102, or the second side 104 of the surface functionalized substrate 10, or both may be modified to include one or more functional groups. As shown in FIGS. 5A, 5B, 5C, 6A, 8A, and 8B, in some other embodiments, at least a portion of either the first side 102, or the second side 104 of the surface functionalized substrate 10, or both may be modified to include one or more functional groups.

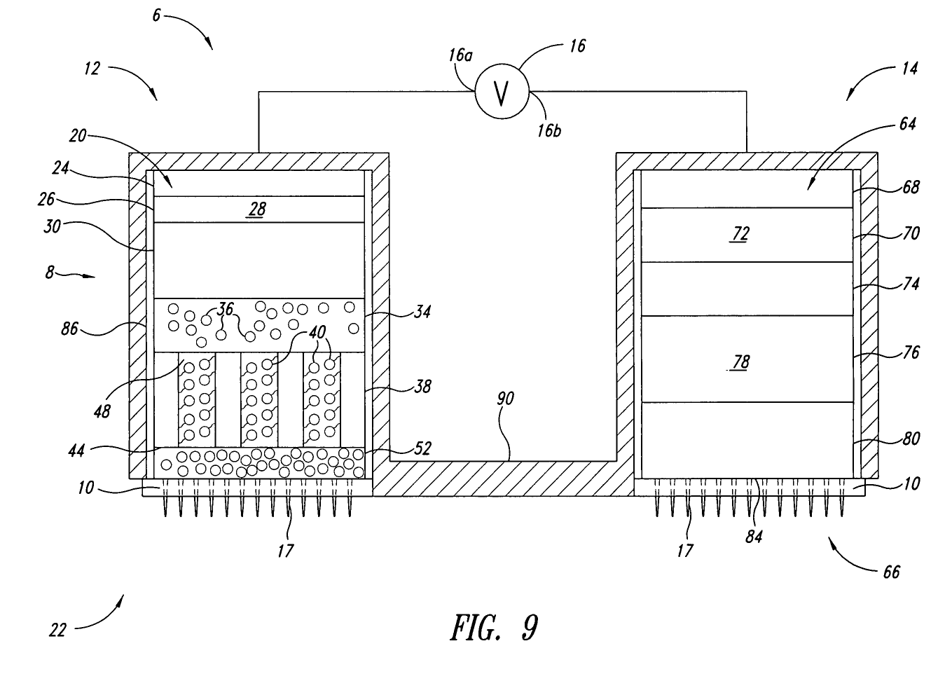

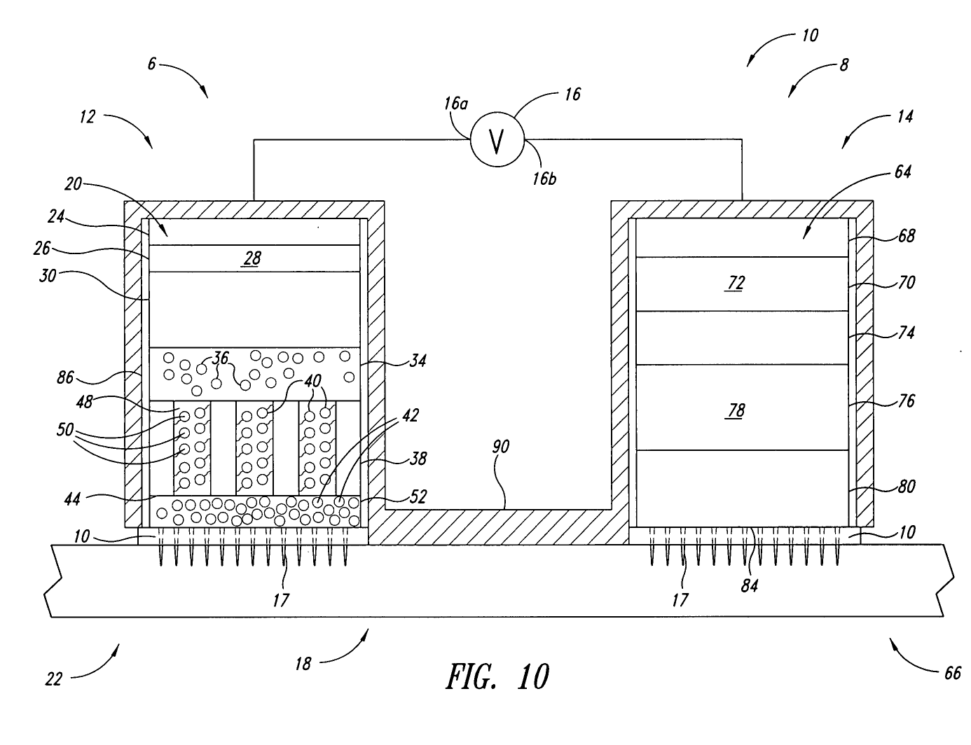

As shown in FIGS. 9 and 10, the iontophoresic delivery device 8 may include active and counter electrode assemblies 12, 14, respectively, and an integrated power source 16, and one or more surface functionalized substrates 10 including a plurality of microneedles 17. The active and counter electrode assemblies 12, 14, are electrically coupleable to the integrated power source 16 to supply an active agent contained in the active electrode assembly 12, via iontophoresis, to a biological interface 18 (e.g., a portion of skin or mucous membrane).

The active electrode assembly 12 may further comprise, from an interior 20 to an exterior 22 of the active electrode assembly 12: an active electrode element 24, an electrolyte reservoir 26 storing an electrolyte 28, an inner ion selective membrane 30, an inner active agent reservoir 34, storing one or more active agents 36, an optional outermost ion selective membrane 38 that optionally caches additional active agents 40, an optional further active agent 42 carried by an outer surface 44 of the outermost ion selective membrane 38, and one or more functionalized substrates 10 including a plurality of outwardly projecting microneedles 17. The active electrode assembly 12 may further comprise an optional outer release liner (not shown).

In some embodiments, one or more active agents 36, 40, 42 are loaded in the at least one active agent reservoir 34. In some embodiments, the one or more active agents 36, 40, 42 are selected from cationic, anionic, ionizable, or neutral active agents. In some embodiments, the one or more active agents include an analgesic. In some embodiments, the one or more active agents 36, 40, 42 take the form of cationic drugs, and the one or more functional groups take the form of negatively charged functional groups.

The surface functionalized substrate 10 may be positioned between the active electrode assembly 12 and the biological interface 10. In some embodiments, the at least one active electrode element 20 is operable to provide an electromotive force to drive an active agent 36, 40, 42 from the at least one active agent reservoir 34, through the plurality of microneedles 106, and to the biological interface 18.

Referring to FIGS. 9 and 10, the active electrode assembly 12 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological surface 18. Each of the above elements or structures will be discussed in detail below.

The active electrode element 24 is electrically coupled to a first pole 16a of the power source 16 and positioned in the active electrode assembly 12 to apply an electromotive force to transport the active agent 36, 40, 42 via various other components of the active electrode assembly 12. Under ordinary use conditions, the magnitude of the applied electromotive force is generally that required to deliver the one or more active agents according to a therapeutic effective dosage protocol. In some embodiments, the magnitude is selected such that it meets or exceeds the ordinary use operating electrochemical potential of the iontophoresis delivery device 8.

The active electrode element 24 may take a variety of forms. In one embodiment, the active electrode element 24 may advantageously take the form of a carbon-based active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 28 may take the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 9 and 10, take the form of an ion exchange membrane having pores 48 (only one called out in FIGS. 9 and 10 for sake of clarity of illustration) of the ion selective membrane 38 including ion exchange material or groups 50 (only three called out in FIGS. 9 and 10 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner to retain the active agent 40 until the outer release liner is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner may generally be positioned overlying or covering further active agent 42 carried by the outer surface 44 of the outermost ion selective membrane 38. The outer release liner may protect the further active agent 42 and/or outermost ion selective membrane 38 during storage, prior to application of an electromotive force or current. The outer release liner may be a selectively releasable liner made of waterproof material, such as release liners commonly associated with pressure sensitive adhesives.

An interface coupling medium (not shown) may be employed between the electrode assembly and the biological interface 18. The interface coupling medium may, for example, take the form of an adhesive and/or gel. The gel may, for example, take the form of a hydrating gel. Selection of suitable bioadhesive gels is within the knowledge of one skilled in the art.

In the embodiment illustrated in FIGS. 9 and 10, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner (not shown).

The counter electrode element 68 is electrically coupled to a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes are discussed above.

Alternatively, the outermost ion selective membrane 80 may take the form of a semi-permeable membrane that substantially passes and/or blocks ions based on size or molecular weight of the ion.

The outer release liner (not shown) may generally be positioned overlying or covering an outer surface 84 of the outermost ion selective membrane 80. The outer release liner may protect the outermost ion selective membrane 80 during storage, prior to application of an electromotive force or current. The outer release liner may be a selectively releasable liner made of waterproof material, such as release liners commonly associated with pressure sensitive adhesives. In some embodiments, the outer release liner may be coextensive with the outer release liner (not shown) of the active electrode assembly 12.

The iontophoresis device 8 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 10, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may, for example, take the form of an adhesive and/or gel. The gel may, for example, take the form of a hydrating gel or a hydrogel. If used, the interface-coupling medium should be permeable by the active agent 36, 40, 42.

In some embodiments, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively electrically coupled to the active and counter electrode assemblies 12, 14 via a control circuit, for example, via carbon fiber ribbons. The iontophoresis device 8 may include discrete and/or integrated circuit elements to control the voltage, current, and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more cationic or anionic drugs or other therapeutic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides or nucleic acids. In other embodiments, the active agent may be mixed with another agent to form a complex capable of being transported across the biological interface via one of the motive methods described above.

In some embodiments, the transdermal drug delivery system 6 includes an iontophoretic drug delivery device 8 for providing transdermal delivery of one or more therapeutic active agents 36, 40, 42 to a biological interface 10. The delivery device 8 includes active electrode assembly 12 including at least one active agent reservoir and at least one active electrode element operable to provide an electromotive force to drive an active agent from the at least one active agent reservoir. The delivery device 8 further includes a surface functionalized substrate 10 in fluidic communication with the active electrode assembly 12 and positioned between the active electrode assembly 12 and the biological interface 18. The surface functionalized substrate 10 includes a first side 102 and a second side 104 opposing the first side 102. The first side 102 includes a plurality of microneedles 106 projecting outwardly. Each microneedle 106 having a channel 116, and an inner 114 and outer surface 112. The channel 116 is operable for providing fluidic communication between the first and the second sides 102, 104 of the surface functionalized substrate 10. In some embodiments, at least one of the inner surface 114 or the outer surface 112 is modified to include a sufficient amount of one or more functional groups to increase an electrophoretic mobility of the one or more active agents 36, 40, 42, through the plurality of microneedles 106, and to the biological interface 18. In some embodiments, the one or more functional groups are selected from charge functional groups, hydrophobic functional groups, hydrophilic functional groups, chemically reactive functional groups, organofunctional group, water-wettable groups, and the like.

The surface functionalized substrate 10 may further include an exterior surface 102a and interior surface 104a, the inner surface 114 of the plurality of microneedles 106 forming a substantial portion of the interior surface 104a of the surface functionalized substrate 10, wherein at least one of the interior or the exterior surfaces 104a, 102a of the surface functionalized substrate comprises silicon dioxide. In some embodiments, the surface functionalized substrate 10 may further comprises a metallic coating. In some other embodiments, the surface functionalized substrate 10 may further comprises a gold coating.

The delivery device 8 may include a counter electrode assembly 14 including at least one counter electrode element 68, and a power source 16 electrically coupled to the at least one active and the at least one counter electrode elements 20, 68. In some embodiments, the iontophoretic drug delivery 8 may further include one or more active agents 36, 40, 42 loaded in the at least one active agent reservoir 34.

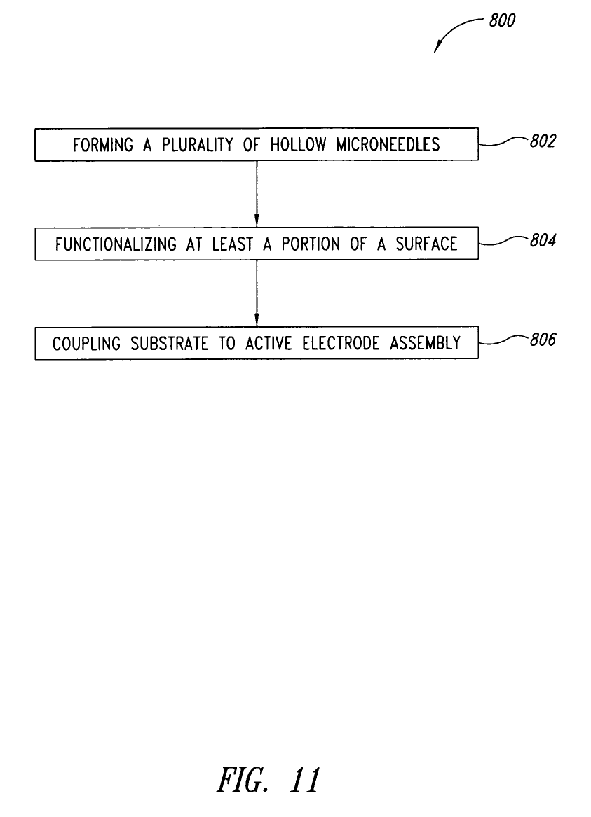

FIG. 11 shows an exemplary method 800 of forming an iontophoretic drug delivery device 8.

At 802, the method 800 includes forming a plurality of hollow microneedles 106, having an interior 114 and an exterior 112 surface, on a substrate 10 having a first side 102 and a second side 104 opposing the first side 102. The plurality of hollow microneedles 106 is substantially formed on the first side 102 of the substrate 10. As previously noted, there are many techniques for fabricating the microneedles 106. On exemplary technique involves forming the microneedles 106 on a silicon dioxide substrate. See for example Rodriquez et al., Fabrication of Silicon Oxide Microneedles from Macroporous Silicon, E-MRS Fall Meeting 2004: Book of Abstracts, pg. 38 (2004). First a series of channels are formed in an n-type silicon substrate 10a using photo-assisted electrochemical etching, in low concentration hydrofluoric (HF) acid. A lithographical pattern is used to define the distances between channels 116. The etched channels 116 are oxidized and the remaining microneedles structures 106 are formed using backside tetra methyl ammonium hydroxide (TMAH) etching. The silicon oxide at the distal end 108 of microneedles 106 is etched in buffered HF acid. The physical characteristics (e.g., shape, interior or exterior diameter, length, and the like) of the resulting microneedles 106 can further be modified. For example, the average diameter of the inner channel 116 of the microneedles 106 may be adjusted by controlling the thickness of SiO₂ on the outer and/or the inner surfaces 112, 114. An outside diameter of the microneedles 106 fabricated by this method can range from about less than 1 μm to about 50 μm.

In some embodiments forming a plurality of hollow microneedles 106 includes forming a photoresist mask for patterning the exterior surface 112 of the plurality of hollow microneedles 106 on the first side 102 of the substrate 10, and forming a photoresist mask for patterning the interior surface 114 of the plurality of hollow microneedles on the second side 104 of the substrate 10. Forming a plurality of hollow microneedles 106 further includes etching the interior surface 114 of the plurality of the hollow microneedles 106 on the second side 104 of the substrate, and etching the exterior surface 112 of the plurality of the hollow microneedles 106 on the first side of the substrate 10.

At 804, the method includes functionalizing at least the interior surface 114 of the plurality of hollow microneedles 106 to include one or more functional groups. In some embodiments, functionalizing at least the interior surface of the plurality of hollow microneedles includes modifying at least the interior surface 114 of the plurality of hollow microneedles 106 to comprise one or more functional groups selected from charge functional groups, hydrophobic functional groups, hydrophilic functional groups, chemically reactive functional groups, organofunctional groups, water-wettable groups, and the like. In some embodiments, functionalizing at least the interior surface 114 of the plurality of hollow microneedles 106 may include hydrolyzing one or more silane coupling agents comprising at least one functional group to form silanols, and coupling the silanols to at least the interior surface 114 of the plurality of hollow microneedles 106. In some further embodiments, the silane coupling agents are selected from Formula I alkoxysilanes:
(R²)Si(R¹)₃ (Formula I)
wherein, R¹ is selected from a chlorine, an acetoxy, and an alkoxy, and R² is selected from an organofunctional group, an alkyl, an aryl, an amino, a methacryloxy, and an epoxy.

In some further embodiments, functionalizing at least the interior surface 114 of the plurality of hollow microneedles 10 may include providing an effective amount of a functionalizing agent comprising a functional group, and a binding group, and coupling the functionalizing agent to at least the interior surface 114 of the plurality of hollow microneedles 106.

At 806, the method includes physically coupling the substrate to an active electrode assembly 12. The active electrode assembly 12 includes at least one active agent reservoir 34 and at least one active electrode element 20. The at least one active agent reservoir 34 is in fluidic communication with the plurality of hollow microneedles 106, and the at least one active electrode element 20 is operable to provide an electromotive force to drive an active agent 36, 40, 42 from the at least one active agent reservoir 34, through the plurality of hollow microneedles 106, and to the biological interface 18.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein can be applied to other agent delivery systems and devices, not necessarily the exemplary iontophoresis active agent system and devices generally described above. For instance, some embodiments may include additional structure. For example, some embodiments may include a control circuit or subsystem to control a voltage, current, or power applied to the active and counter electrode elements 20, 68. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a drug solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte holding part; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488,970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; Japanese patent application Serial No. 2005-081220, filed Mar. 22, 2005, and U.S. Provisional Patent Application No. 60/722,789, filed Sep. 30, 2005,

As one of skill in the art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. A transdermal drug delivery system for delivering of one or more therapeutic active agents to a biological interface, comprising:
a surface functionalized substrate having a first side and a second side opposing the first side, the surface functionalized substrate comprising a plurality of microneedles projecting outwardly from the first side, each microneedle having an outer surface and an inner surface that forms a channel, the channel operable for providing fluidic communication between the first and the second sides of the surface functionalized substrate, at least one of the inner surface or the outer surface comprising one or more functional groups.

2. The transdermal drug delivery system of claim 1, wherein the one or more functional groups are selected from charge functional groups, hydrophobic functional groups, hydrophilic functional groups, chemically reactive functional groups, organofunctional group, and bio-compatible groups.

3. The transdermal drug delivery system of claim 1, wherein the one or more functional groups are selected from the following Formula I alkoxysilanes:
(R²)Si(R¹)₃ (Formula I)
wherein,
R¹ is selected from a chlorine, an acetoxy and alkoxy; and
R² is selected from an organofunctional group, an alkyl, an aryl, an amino, a methacryloxy, and an epoxy.

4. The transdermal drug delivery system of claim 1, wherein the surface functionalized substrate comprises at least one material selected from ceramics, metals, polymers, molded plastics, and superconductor wafers.

5. The transdermal drug delivery system of claim 1, wherein the surface functionalized substrate comprises at least one material selected from elastomers, epoxy photoresist, glass, glass polymers, glass/polymer materials, chromium, cobalt, gold, molybdenum, nickel, stainless steel, titanium, tungsten steel, biodegradable polymers, non-biodegradable polymers, organic polymers, inorganic polymers, silicon, silicon dioxide, polysilicon, silicon-based organic polymers, silicon rubbers, superconducting materials, or combinations, composites, and alloys thereof.

6. The transdermal drug delivery system of claim 1, further comprising:
an active electrode assembly including at least one active electrode element; and
a counter electrode assembly including at least one counter electrode element.

7. The transdermal drug delivery system of claim 6, wherein the active electrode assembly further comprises:
at least one active agent reservoir; and
wherein the surface functionalized substrate is positioned between the active electrode assembly and the biological interface, and the at least one active electrode element is operable to provide an electromotive force to drive an active agent from the at least one active agent reservoir, through the plurality of microneedles, and to the biological interface.

8. The transdermal drug delivery system of claim 7, further comprising:
one or more active agents loaded in the at least one active agent reservoir.

9. The transdermal drug delivery system of claim 7, wherein the one or more therapeutic active agents are selected from cationic, anionic, ionizable, or neutral active agents.

10. The transdermal drug delivery device of claim 7, wherein the one or more active agents are selected from analgesics, anesthetics, anesthetics vaccines, antibiotics, adjuvants, immunological adjuvants, immunogens, tolerogens, allergens, toll-like receptor agonists, toll-like receptor antagonists, immuno-modulators, immuno-response agents, immuno-stimulators, specific immuno-stimulators, non-specific immuno-stimulators, and immuno-suppressants, or combinations thereof.

11. The transdermal drug delivery system of claim 7, wherein the one or more therapeutic active agents are cationic, and the one or more functional groups take the form of negatively charged functional groups.

12. The transdermal drug delivery system of claim 6, further comprising:
a power source electrically coupled to the at least one active and the at least one counter electrode elements.

13. The transdermal drug delivery system 12 wherein the power source comprises at least one of a chemical battery cell, super- or ultra-capacitor, a fuel cell, a secondary cell, a thin film secondary cell, a button cell, a lithium ion cell, zinc air cell, and a nickel metal hydride cell.

14. A microneedle structure, comprising:
a substrate having an exterior and an interior surface, a first side, and a second side opposing the first side; and
a plurality of microneedles projecting outwardly from the first side of the substrate, each microneedle having a proximate and a distal end, an outer surface and an inner surface forming a channel exiting between the proximate and the distal ends to provided fluid communication there between;
wherein at least the inner surface of the microneedles is modified with one or more functional groups.

15. The microneedle structure of claim 14 wherein each microneedle is substantially hollow, and each microneedle is substantially in the form of a frusto-conical annulus.

16. The microneedle structure of claim 14 wherein the plurality of microneedles is integrally formed from the substrate.

17. The microneedle structure of claim 14 wherein the plurality of microneedles are arranged in the form of an array.

18. The microneedle structure of claim 14 wherein at least the interior surface of the substrate is modified with a sufficient amount of one or more functional groups.

19. The microneedle structure of claim 14, wherein the substrate comprises at least one material selected from ceramics, elastomers, epoxy photoresist, glass, glass polymers, glass/polymer materials, metals, chromium, cobalt, gold, molybdenum, nickel, stainless steel, titanium, tungsten steel, molded plastics, polymers, biodegradable polymers, non-biodegradable polymers, organic polymers, inorganic polymers, silicon, silicon dioxide, polysilicon, silicon-based organic polymers, silicon rubbers, superconducting materials, superconducting wafers, or combinations, composites, and alloys thereof.

20. The microneedle structure of claim 14 wherein the one or more functional groups are selected from charge functional groups, hydrophobic functional groups, hydrophilic functional groups, chemically reactive functional groups, organofunctional group, and bio-compatible groups.

21. A method of forming an iontophoretic drug delivery device for providing transdermal delivery of one or more therapeutic active agents to a biological interface, comprising:
forming a plurality of hollow microneedles, having an interior and an exterior surface on a substrate having a first side and a second side opposing the first side, the plurality of hollow microneedles substantially formed on the first side of the substrate;
functionalizing at least the interior surface of the plurality of hollow microneedles to include one or more functional groups; and
physically coupling the substrate to an active electrode assembly, the active electrode assembly including at least one active agent reservoir and at least one active electrode element, the at least one active agent reservoir in fluidic communication with the plurality of hollow microneedles, the at least one active electrode element operable to provide an electromotive force to drive an active agent from the at least one active agent reservoir, through the plurality of hollow microneedles, and to the biological interface.

22. The method of claim 21 wherein forming a plurality of hollow microneedles comprises:
forming a photoresist mask for patterning the exterior surface of the plurality of hollow microneedles on the first side of the substrate;
forming a photoresist mask for patterning the interior surface of the plurality of hollow microneedles on the second side of the substrate;
etching the interior surface of the plurality of the hollow microneedles on the second side of the substrate; and
etching the exterior surface of the plurality of the hollow microneedles on the first side of the substrate.

23. The method of claim 21, wherein functionalizing at least the interior surface of the plurality of hollow microneedles comprises:
modifying at least the interior surface of the plurality of hollow microneedles to comprise one or more functional groups selected from charge functional groups, hydrophobic functional groups, hydrophilic functional groups, chemically reactive functional groups, organofunctional groups, and water-wettable groups.

24. The method of claim 21 wherein functionalizing at least the interior surface of the plurality of hollow microneedles comprises:
hydrolyzing one or more silane coupling agents comprising at least one functional group to form silanols; and
coupling the silanols to at least the interior surface of the plurality of hollow microneedles.

25. The method of claim 24 wherein the silane coupling agents are selected from Formula I alkoxysilanes:
(R²)Si(R¹)₃ (Formula I)
wherein,
R¹ is selected from a chlorine, an acetoxy, and an alkoxy; and
R² is selected from an organofunctional group, an alkyl, an aryl, an amino, a methacryloxy, and an epoxy.

26. The method of claim 21 wherein functionalizing at least the interior surface of the plurality of hollow microneedles comprises:
providing an effective amount of a functionalizing agent comprising a functional group, and a binding group; and
coupling the functionalizing agent to at least the interior surface of the plurality of hollow microneedles.
